(19) United States
(12) Patent Application Publication
(10) Pub. No.: US 2024/0247202 A1
(43) Pub. Date: Jul. 25, 2024

(54) PROCESS FOR PRODUCING SYNTHETIC HYDROCARBONS FROM BIOMASS

(71) Applicants: EXPANDER ENERGY INC., Calgary (CA); ATOMIC ENERGY OF CANADA LIMITED, Chalk River (CA)

(72) Inventors: Steve KRESNYAK, Calgary (CA); Gord CRAWFORD, Calgary (CA); Sellathurai SUPPIAH, Deep River (CA)

(73) Assignees: EXPANDER ENERGY INC., Calgary, AB (CA); ATOMIC ENERGY OF CANADA LIMITED, Chalk River, ON (CA)

(21) Appl. No.: 18/560,634

(22) PCT Filed: May 12, 2022
(86) PCT No.: PCT/CA2022/050751

§ 371 Date: Nov. 13, 2023

Related U.S. Application Data
(60) Provisional application No. 63/187,493, filed on May 12, 2021.
(60) Provisional application No. 63/334,544, filed on Apr. 25, 2022.

(51) Int. Cl. C10J 3/18; C07C 1/04

(52) U.S. Cl. CPC C10J 3/18; C07C 1/0485; C10J 2300/1659; C10J 2300/1671

(57) ABSTRACT

A process for preparing synthetic hydrocarbons from a biomass feedstock is provided. The process involves electrolysis of steam and/or CO₂, optionally along with a refinery gas in a high temperature co-electrolyzer (HTCE) to produce oxygen and hydrogen and/or enhanced hydrogen rich syngas. The oxygen generated via the electrolysis process is used for partial oxidation of a biomass feedstock in a gasifier to generate a hydrogen lean syngas. The hydrogen lean syngas is mixed with at least a portion of the hydrogen and/or enhanced hydrogen rich syngas generated via the high temperature electrolysis/co-electrolysis to formulate a hydrogen rich syngas. The hydrogen rich syngas is then reacted in a Fischer Tropsch (FT) reactor to produce synthetic hydrocarbons and refinery gas.

### FIELD OF THE INVENTION

The present invention pertains to the field of production of synthetic hydrocarbons from renewable and/or low carbon sources.

### BACKGROUND OF THE INVENTION

The carbon-based fossil fuels such as coal, oil and natural gas are non-renewable resources and of limited supply. Combustion of fossil fuel has caused a rise in atmospheric carbon dioxide concentrations, which are believed to contribute to global climate change. The concern for carbon emissions from fossil fuels has created an increased interest in the development of synthetic fuel sources.

Biofuels are considered viable alternatives to fossil fuels for several reasons. Biofuels are renewable energy sources produced from biomass. One of the advantageous features of the biomass to fuel technology is that it presents a possibility to not only formulate a less carbon intensive pure biosynthetic fuel product, but also make use of waste biomass materials, such as forestry by products, construction and other wood waste products, human waste products, or agriculture feedstock, byproducts and waste products.

The Fischer-Tropsch (FT) process converts hydrogen and carbon monoxide (commonly known as syngas) into liquid hydrocarbons, examples of which include synthetic diesel, naphtha, kerosene, aviation or jet fuel and paraffinic wax. For an effective FT reaction, the molar ratio of the H₂:CO in the syngas is required to be approximately 2:1.

Several biomass to liquid processes have been developed, that involve thermal gasification of biomass to generate syngas and utilizing same in the FT reaction.

As is well known the art, gasification of biomass results in a hydrogen lean syngas having H₂:CO molar ratio of approximately 1:1. As a result, biomass to liquid processes involving the FT reaction require the incorporation of water gas shift (WGS) reaction, or generation of separate hydrogen rich syngas streams using gas/methane reformers, such as a steam methane reformer (SMR) and/or an autothermal reformer (ATR), to supplement the hydrogen lean syngas.

Historically, water gas shift (WGS) processing has been used, but this process is extremely wasteful and uneconomic. The water gas shift reaction is a shift from the CO to CO₂ to create a hydrogen rich syngas, which involves adding water vapor to the hydrogen lean syngas, wherein water reacts with carbon monoxide to form carbon dioxide and additional hydrogen. The WGS reaction therefore requires heat and generates undesirable CO₂.

Reforming of natural gas via SMR and/or ATR also requires heat addition for combustion of natural gas, a non-renewable resource.

A Biomass to Liquids (BTL) process such as disclosed in WO2012106795 incorporates biomass gasification and natural gas reforming to provide hydrocarbon liquid products with lower carbon intensity (CI) than petroleum fuels (reduction of over 40%). However, this process is also dependent upon non-renewable feedstock (i.e. natural gas).

Integration of biomass gasification and water electrolysis has been used for the production of hydrogen, wherein water electrolysis is conducted to supply oxygen for a biomass gasifier and the side stream of hydrogen is used to supplement the hydrogen stream from the gasifier. The process involves a water gas shift reaction to convert hydrogen lean syngas obtained from a gasifier into a hydrogen rich syngas, which results in the production of CO₂, which is rejected to atmosphere (International Journal of Hydrogen Energy 34 (2009) 772-782). This article also concluded that use of electrolysis for hydrogen production is not cost effective.

Integration of biomass gasification and water electrolysis to generate a hydrogen rich syngas has been disclosed by Mckellar et al., in International Mechanical Engineering Congress and Exposition, Oct. 31-Nov. 6, 2008. This article also discloses that the process efficiency can vary significantly depending on biomass inputs and gasifier temperature and efforts to increase efficiency results in the formation of more CO₂.

Accordingly, there is a need for an improved carbon efficient biomass to liquids (BTL) process for producing 100% biosynthesized hydrocarbons, which does not depend on non-renewable feedstock, and which can utilize renewable and/or low carbon energy source (such as wind, solar, hydro, nuclear, etc.) to produce oxygen for biomass oxidation and produce hydrogen and/or enhanced hydrogen rich syngas for supplementing the hydrogen lean syngas obtained from biomass.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a process for production of biosynthetic hydrocarbons from renewable and/or low carbon sources.

In accordance with an aspect of the present invention, there is provided a process for preparing synthetic hydrocarbons from a biomass feedstock, which comprises:
a) electrolyzing steam and CO₂ in a high temperature co-electrolyzer to produce oxygen, enhanced hydrogen rich syngas and heat energy;
b) feeding the oxygen generated in step a), and the biomass feedstock into a gasifier, and gasifying the feedstock under partial oxidation reaction conditions to generate a hydrogen lean syngas, wherein the biomass feedstock optionally undergoes a step of removing excess moisture prior to being fed to the gasifier;
c) cooling the hydrogen lean syngas obtained in step b) to generate process water and heat energy;
d) adding at least a portion of the enhanced hydrogen rich syngas generated in step a) to the hydrogen lean syngas to formulate hydrogen rich syngas;
e) reacting the hydrogen rich syngas in a Fischer Tropsch (FT) reactor to produce the synthetic hydrocarbons, process water, heat energy and refinery gas.

In accordance with an aspect of the present invention, there is provided a process for preparing synthetic hydrocarbons from a biomass feedstock, which comprises:
a) electrolyzing steam in a high temperature electrolyzer or co-electrolyzer to produce oxygen, hydrogen, and heat energy;
b) feeding the oxygen generated in step a), and the biomass feedstock into a gasifier, and gasifying the feedstock under partial oxidation reaction conditions to generate a hydrogen lean syngas, wherein the biomass feedstock optionally undergoes a step of removing excess moisture prior to being fed to the gasifier;
c) cooling the hydrogen lean syngas obtained in step b) to generate process water and heat energy;
d) adding at least a portion of the hydrogen generated in step a) to the hydrogen lean syngas to formulate hydrogen rich syngas;
e) reacting the hydrogen rich syngas in a Fischer Tropsch (FT) reactor to produce the biosynthetic hydrocarbons, process water, heat energy and refinery gas.

The process may further include recycling at least a portion of process water generated during the cooling of the hydrogen lean syngas in step c) and/or generated in the FT reaction in step e) for generating steam for the high temperature electrolysis/co-electrolysis; recycling at least a portion of the refinery gas produced in the process to the co-electrolyzer, and/or recycling at least a portion of the heat energy produced in the process for generating steam for high temperature electrolysis/co-electrolysis.

### BRIEF DESCRIPTION OF THE FIGURES

The invention will now be described by way of an exemplary embodiment with reference to the accompanying simplified, flow diagrams. In the drawings:

FIG. 1 depicts a flow diagram of a conventional biomass to liquids process;

FIG. 2 depicts a flow diagram of a biomass to liquids process in accordance with an embodiment of the present invention.

FIG. 3 depicts a flow diagram of a biomass to liquids process in accordance with an embodiment of the present invention.

FIG. 4 depicts a flow diagram of a biomass to liquids process in accordance with an embodiment of the present invention.

### DETAILED DESCRIPTION OF THE INVENTION

Unless defined otherwise, all technical and scientific terms used herein have the same meaning as commonly understood by one of ordinary skill in the art to which this invention belongs.

As used herein, the term “syngas” is an abbreviation for “synthesis gas”, which is a mixture comprising hydrogen, carbon monoxide, and some carbon dioxide.

As used herein, the term “hydrogen lean syngas” refers to syngas having H₂:CO molar ratio of about 1:1, such as 0.5:1 to 1.2:1.

As used herein, the term “hydrogen rich syngas” refers to syngas having H₂:CO molar ratio of about 2:1, such as 1.8:1 to 2.2:1, which is the desired optimum ratio for use in Fischer-Tropsch reaction.

As used herein, the term “enhanced hydrogen rich syngas” refers to syngas having H₂:CO molar ratio of greater than 2.2:1, such as 2.3:1 to 7:1, which can be used to mix with a hydrogen lean syngas to form the optimum ratio for use in Fischer-Tropsch reaction, and which contains excess hydrogen for other internal or external use.

As used herein, the term “electrolysis” refers to the process of using electricity to split/convert water into hydrogen and oxygen.

As used herein, the term “co-electrolysis” refers to the process of using electricity to convert water and CO₂ and/or refinery gas into hydrogen rich syngas or enhanced hydrogen rich syngas, and oxygen.

As used herein, the term “high temperature co-electrolysis” refers to the process of using electricity to convert steam and CO₂ and/or refinery gas into hydrogen rich syngas and oxygen at temperatures greater than 100° C., and preferred at 700° C. to 1000° C.

As used herein, the term “refinery gas” refers to vapour streams from one or more unit operations, such as syngas treatment, FT reaction, FT product upgrading, hydrogen separation, etc., (which may contain H₂, CO, CO₂, hydrocarbons, water and/or inert gases such as nitrogen and argon), and which is re-used in the process.

As used herein, the term “off gas” refers to vapour streams recovered from unit operations, such as hydrogen separation, hydrocarbons upgrading, etc., which may contain H₂, CO, CO₂, hydrocarbons, water, and/or inert gases such as nitrogen and argon.

As used herein, the term “tail gas” refers to vapour streams recovered from FT unit operations, which may contain H₂, CO, CO₂, hydrocarbons, water, and/or inert gases such as nitrogen and argon.

As used herein, the term “about” refers to a +/−10% variation from the nominal value. It is to be understood that such a variation is always included in a given value provided herein, whether or not it is specifically referred to.

The present invention relates to a process for production of biosynthetic hydrocarbon from low carbon and/or renewable sources, i.e. biomass, water, CO₂ and/or or refinery gas, and electricity.

The present application provides an improved biomass to liquid process for preparing synthetic hydrocarbons, which utilizes low carbon and/or renewable energy to produce oxygen, and hydrogen and/or enhanced hydrogen rich syngas. The oxygen is utilized for efficient operation of the biomass gasifier and the hydrogen and/or enhanced hydrogen rich syngas is utilized for the production of a tar free hydrogen rich syngas suitable for Fischer Tropsch (FT) conversions to obtain synthetic hydrocarbons, including transportation fuels.

The inventors of the present application have found that integration of high temperature electrolysis/co-electrolysis, biomass gasification, and FT reaction for production of synthetic hydrocarbons results in near stoichiometric conditions, wherein substantially all of the hydrogen/enhanced hydrogen rich syngas, and oxygen generated via the high temperature electrolysis/co-electrolysis is efficiently consumed in the process. In addition, recycling at least a portion of the refinery gas produced in the process to the co-electrolyzer, recycling at least a portion of the heat energy produced in the process for generating steam for high temperature electrolysis, and/or recycling at least a portion of the process water produced in the process for generating steam, along with other optional recycling steps as described herein, can result in a highly carbon efficient and economically viable process.

The process of the present application does not include the water gas shift reaction or natural gas reforming, thereby reducing the carbon foot print and dependence on non-renewable feedstocks (e.g. natural gas). Low carbon renewable hydro/solar/wind sourced electricity (which is plentiful and inexpensive in many regions) or low carbon nuclear power can be utilized to eliminate the need for a non-renewable source, such as natural gas.

The process of the present invention involves electrolysis of steam and/or CO₂, optionally along with a refinery gas in a high temperature electrolyzer/co-electrolyzer (HTCE) to produce oxygen and hydrogen and/or enhanced hydrogen rich syngas. The oxygen generated via the electrolysis process is used for partial oxidation of a biomass feedstock in a gasifier to generate a hydrogen lean syngas. Gasification of biomass results in generation of hot raw syngas, which is fed to a heat exchanger/steam generator to be cooled resulting in the generation of high quality process water for downstream use. The cooled raw hydrogen lean syngas is mixed with at least a portion of the hydrogen and/or enhanced hydrogen rich syngas generated via the high temperature electrolysis/co-electrolysis to formulate a hydrogen rich syngas. The hydrogen rich syngas is then reacted in a Fischer Tropsch (FT) reactor to produce synthetic hydrocarbons and refinery gas.

Process water generated during the cooling of the hydrogen lean syngas and/or generated in the FT reaction is treated to obtain high quality process water and recycled for generating steam for the high temperature electrolysis/co-electrolysis step, thereby minimizing/eliminating the amount of water required from an external source, eventually using the recycled water as primary source for the electrolysis process.

In some embodiments, the process water generated during the cooling of the hydrogen lean syngas optionally in combination with process water generated in the FT reaction is recycled for generating steam for the high temperature electrolysis/co-electrolysis step.

In some embodiments, a portion of the refinery gas produced in the process is recycled to the high temperature electrolyzer/co-electrolyzer, at least a portion of the heat energy produced in the process is recycled for generating steam for the high temperature electrolysis-co-electrolysis, and/or at least a portion of the process water produced in the process is recycled for generating steam for the high temperature electrolysis-co-electrolysis.

Any suitable high temperature steam electrolyzer (HTSE) can be selected to conduct the electrolysis step and is preferred over the conventional electrolyzer due to its 30% lower electrical requirement. When CO₂ and/or Refinery Gases are used with the steam as feed to the HTSE, the combination is referred to as a high temperature co-electrolyzer (HTCE). A suitable temperature and/or pressure for the co-electrolysis is selected as appropriate for the type of co-electrolyzer used.

In some embodiments, the electrolysis/co-electrolysis step can be carried out at a temperature from about 100° C. to about 1000° C. In some embodiments, the high temperature co-electrolysis step is carried out at temperature above 250° C. to about 850° C.

In some embodiments, the co-electrolysis step can be carried out at a pressure up to 50 bar.

The advances in the design of Solid Oxide Electrolytic Cells (SOEC) and similar electrolyzer devices enables the efficient reduction of steam and carbon containing off gases, such as CO₂ and CH₄ to be reformed in the cathode side to produce high quality enhanced hydrogen rich syngas.

Non-limiting examples of carbon dioxide sources include captured atmospheric carbon dioxide, emissions from industrial processes, such as cement manufacturing, etc., and carbon rich streams generated in the process.

In some embodiments, the process comprises removing excess moisture from the biomass feedstock to achieve a desired water content level prior to feeding the feedstock to the biomass gasifier. Excess moisture from the biomass feedstock can be removed by subjecting the initial feedstock to a biomass dryer. The desired water content level in the present process is less than 20%, preferably about 10-15%.

In some embodiments, heat recovered from electrolysis step, cooling of the hydrogen lean syngas, and/or from the FT reactor is recycled for removing excess moisture from the biomass.

The Fischer-Tropsch (FT) reaction is a highly exothermic reaction. At least a portion of energy/heat from the FT reaction, typically in the form of steam, is used in the process described herein, such as to remove excess moisture from the biomass feedstock, to generate power/electricity, and/or to generate steam for electrolysis step.

In some embodiments, the process comprises feeding at least a portion of the steam generated during the FT reaction to recover heat, which is then used to remove excess moisture from the biomass feedstock and/or to generate steam for electrolysis step.

In some embodiments, the process comprises feeding at least a portion of steam generated in the FT reaction to an electricity generator to produce electricity which can be used to supplement electricity for the electrolyzer/co-electrolyzer, and the residual heat after power generation is used to remove excess moisture from the biomass feedstock.

Synthetic hydrocarbons obtained from the FT reaction can be subjected to further upgrading processes to obtain desired products. As is known by those skilled in the art, several hydrocarbon treatment methods can form part of the upgrading step depending on the desired refined products, which are essentially free of sulfur. The resulting diesel may be used to produce environmentally friendly, sulfur-free fuel and/or blending stock for fuels by using as is or blending with higher sulfur fuels created from petroleum sources.

The hydrocarbons recovered from the upgrading process can be further fractionated to obtain products such as naphtha, diesel, kerosene, jet fuel, lube oil, wax, etc.

In some embodiments, a portion of the hydrogen or enhanced hydrogen rich syngas generated via the electrolysis step is subjected to a hydrogen separation operation, such as membrane, pressure swing adsorption (PSA) or absorption operation to generate a high purity hydrogen stream.

In some embodiments, the tail gas produced in the FT reaction, the off gas produced in the FT product fractionation step, the off gas obtained during hydrogen separation operation, or a combination thereof is recycled to the electrolyzer/co-electrolyzer to augment formation of enhanced hydrogen rich syngas.

In some embodiments, the tail gas produced in the FT reaction, the off gas produced in the FT product fractionation step, the off gas obtained during hydrogen separation step, or a combination thereof is recycled to the biomass dryer for removing excess moisture from the biomass feedstock and/or for generating electricity for use in electrolysis.

As is known in the art, electrolysis processes result in generation of heat, which can be recovered. In some embodiments, a portion of the heat generated in the electrolysis step can be transferred to the electrolyzer feed streams. In some embodiments, the process comprises recycling at least a portion of the heat generated in the electrolysis step for removing excess moisture from the biomass feedstock. In some embodiments, a portion of the heat generated in the electrolysis step can be used for generating power for the electrolyzer. In some embodiments, a portion of the heat generated in the electrolysis step can be used for generating heat for the electrolyzer feed streams.

Waste heat from the electrolysis step can be captured through organic Rankine cycle (ORC) and/or Sterling cycle generator technology.

In some embodiments, the hot raw hydrogen lean syngas can be fed to a steam-generating heat exchanger to produce steam. In some embodiments, the process comprises utilizing the steam generated via the heat exchanger to produce electricity to operate the electrolyzer, thereby reducing the amount of electricity from the external source.

In some embodiments, the process further comprises recycling/utilizing at least a portion of the excess heat generated during the gasification step for removing excess moisture from the biomass feedstock.

In some embodiments, the off gas formed during fractionation process is recycled to the biomass dryer for removing excess moisture from the biomass feedstock.

In some embodiments, the heat from the FT reaction, heat from the gasification reaction and the refinery gas generated in the FT-reaction and/or the fractionation process are recycled to the biomass dryer for removing excess moisture from the biomass feedstock.

In some embodiments, the refinery gas from the FT reaction (i.e. tail gas) and/or from the fractionation process (i.e. off gas) can be used as a purge gas to fuel an internal combustion engine or micro-turbine to generate power for the electrolyzer. The waste heat from the internal combustion engine can be captured via waste heat recovery technology.

In some embodiments, the hydrogen lean syngas obtained from the gasifier is subjected to cleaning operation(s) prior to use in the FT reaction to remove syngas contaminants, such as fine ash dust, tars, nitrogen based compounds (NH₃, HCN, etc.), sulfur based compounds (H₂S, COS, etc.), hydrogen halides (HCl, HF, etc.) and trace metals (Na, K, etc.). Such cleaning operations involve scrubbing units and guard units known to those skilled in the art to create a relatively clean syngas suitable for use in a Fischer-Tropsch unit.

In some embodiments, the raw hydrogen lean syngas obtained from the gasification of biomass feedstock or after the cleaning operation(s), is treated to a carbon dioxide removal operation prior to reaction in the FT-reactor. In some embodiments, the separated carbon dioxide is fed to the gasifier as blanket/sealing gas to prevent air ingress. In some embodiments, the separated bio-CO₂ is subjected to compression and dehydration for further utilization or sequestration.

In some embodiments, the tail gas obtained from the FT reaction, the off gas obtained from the product fractionation and/or the hydrogen separation is treated to a carbon dioxide removal operation prior to reaction in the high temperature electrolyzer. In some embodiments, the separated carbon dioxide is fed to the gasifier as blanket/sealing gas to prevent air ingress. In some embodiments, the separated bio-CO₂ is subjected to compression and dehydration for further utilization or sequestration.

The biogenic CO₂ is extracted from the atmosphere by the trees, and therefore this process would directly contribute to the direct reduction of green-house gases (GHG) from the atmosphere.

In some embodiments, the raw hydrogen lean syngas obtained from the gasification of biomass feedstock or after the cleaning operation(s), is treated to a carbon dioxide removal operation prior to reaction in the FT-reactor. In some embodiments, the separated carbon dioxide is fed to the gasifier as blanket/sealing gas to prevent air ingress. In some embodiments, the separated bio-CO₂ is subjected to compression and dehydration for further utilization or sequestration.

In some embodiments, a portion of the hydrogen generated in the hydrogen separation step is fed to the hydro-processing operation. Off gases generated during hydro-processing operation(s) can also be used in power generation.

In accordance with an embodiment of the present invention, the process for preparing synthetic hydrocarbons from a biomass feedstock, comprises: a) electrolyzing steam in a high temperature co-electrolyzer to produce oxygen, hydrogen, and heat energy; b) feeding the oxygen generated in step a), and the biomass feedstock into a gasifier, and gasifying the feedstock under partial oxidation reaction conditions to generate a hydrogen lean syngas, wherein the biomass feedstock optionally undergoes a step of removing excess moisture prior to being fed to the gasifier; c) cooling the hydrogen lean syngas obtained in step b) to generate process water and heat energy; d) adding at least a portion of the hydrogen generated in step a) to the hydrogen lean syngas to formulate hydrogen rich syngas; e) reacting the hydrogen rich syngas in a Fischer Tropsch (FT) reactor to produce the biosynthetic hydrocarbons, process water, heat energy and refinery gas.

In some embodiments, the process further comprises recycling at least a portion of the refinery gas produced in step e) to the co-electrolyzer to generate enhanced hydrogen rich syngas, and adding a portion of the enhanced hydrogen rich syngas in step d) to augment formulation of the hydrogen rich syngas; b) recycling at least a portion of the heat energy produced in step a), produced in step c), produced in step e), or a combination thereof, for generating steam for use in step a); and/or c) recycling at least a portion of the process water produced in step c), produced in step e), or both for use in step a).

In some embodiments, the process further comprises adding CO₂ to the co-electrolyzer to augment production of the enhanced hydrogen rich syngas. The CO₂ is from an external source or obtained by treating the hydrogen lean syngas and/or the refinery gas to a carbon dioxide separation operation. In some embodiments, the process further comprises compressing at least a portion of the separated carbon dioxide to generate high purity carbon dioxide for sequestration or market.

In some embodiments, the process further comprises recycling at least a portion of the heat energy produced in step a), produced in step c), produced in step e), or a combination thereof, for removing excess moisture from the biomass prior, for generating electric power for use in step a), or both.

In some embodiments, the process further comprises recycling at least a portion of the refinery gas produced in step e) for removing excess moisture from the biomass, generating electric power for use in step a), or both.

In some embodiments, the process further comprises fractionating the synthesized hydrocarbons, wherein additional refinery gas is generated, and the process further comprises recycling at least a portion of the additional refinery gas: i) to the co-electrolyzer, ii) for removing excess moisture from the biomass i; iii) for generating electric power for use in step a); or iv) a combination thereof.

In some embodiments, the process further comprises recycling at least a portion of heat energy generated in step a) and/or at least a portion of excess heat generated in step c) for removing excess moisture from the biomass feedstock.

In some embodiments, the heat energy generated in step c) is in the form of steam, and the process further comprises recycling at least a portion of steam to an electricity generator to produce electricity to supplement electricity for the co-electrolyzer.

In some embodiments, the heat energy generated in step e) is in the form of steam, and the process further comprises feeding at least a portion of steam to an electricity generator to produce electricity to supplement electricity for the co-electrolyzer and/or to remove excess moisture from the biomass.

The process further includes subjecting the synthesized hydrocarbons to one or more upgrading operations.

In some embodiments, the process further comprises treating a portion of the enhanced hydrogen rich syngas to generate a high purity hydrogen stream.

In some embodiments, the process comprises recovering and recycling excess water removed from the biomass for supplementing water for generating steam for use in electrolysis step.

In accordance with another embodiment of the present invention, the process for preparing synthetic hydrocarbons from a biomass feedstock, comprises: a) electrolyzing steam and CO₂ in a high temperature co-electrolyzer to produce oxygen, enhanced hydrogen rich syngas and heat energy; b) feeding the oxygen generated in step a), and the biomass feedstock into a gasifier, and gasifying the feedstock under partial oxidation reaction conditions to generate a hydrogen lean syngas, wherein the biomass feedstock optionally undergoes a step of removing excess moisture prior to being fed to the gasifier; c) cooling the hydrogen lean syngas obtained in step b) to generate process water and heat energy; d) adding at least a portion of the enhanced hydrogen rich syngas generated in step a) to the hydrogen lean syngas to formulate hydrogen rich syngas; e) reacting the hydrogen rich syngas in a Fischer Tropsch (FT) reactor to produce the synthetic hydrocarbons, process water, heat energy and refinery gas.

In some embodiments, the process further comprises: a) recycling at least a portion of the refinery gas produced in step e) to the co-electrolyzer to augment production of the enhanced hydrogen rich syngas; b) recycling at least a portion of the heat energy produced in step a), produced in step c), produced in step e), or a combination thereof, for generating steam for use in step a); and/or c) recycling at least a portion of the process water produced in step c), produced in step e), or both for use in step a).

In some embodiments, the process further comprises recycling at least a portion of the heat energy produced in step a), produced in step c), produced in step e), or a combination thereof, for removing excess moisture from the biomass, for generating electric power for use in step a), or both.

In some embodiments, the process further comprises recycling at least a portion of the refinery gas produced in step e) for removing excess moisture from the biomass, generating electric power for use in step a), or both.

In some embodiments, the hydrogen lean syngas is treated to a carbon dioxide separation operation prior to the reaction in the FT-reactor, and the process further comprises i) adding at least a portion of the separated carbon dioxide to the co-electrolyzer, and/or ii) compressing at least a portion of the separated carbon dioxide to generate high purity carbon dioxide for sequestration or market.

In some embodiments, the refinery gas generated in step e) is treated to a carbon dioxide separation operation, and the process further comprises adding at least a portion of the separated carbon dioxide to the co-electrolyzer, and/or compressing at least a portion of the separated carbon dioxide to generate high purity carbon dioxide for sequestration or market.

In some embodiments, the process further comprises fractionating the synthesized hydrocarbons, wherein additional refinery gas is generated, and the process further comprises recycling at least a portion of the additional refinery gas: i) to the co-electrolyzer to augment the production of the enhanced hydrogen rich syngas, ii) for removing excess moisture from the biomass in step b); iii) for generating electric power for use in step a); or iv) a combination thereof. In some embodiments, the additional refinery gas is treated to a carbon dioxide separation operation, and the process further comprises adding at least a portion of the separated carbon dioxide to the co-electrolyzer, and/or compressing at least a portion of the separated carbon dioxide to generate high purity carbon dioxide for sequestration or market.

In some embodiments, the process further comprises recycling at least a portion of heat energy generated in step a) and/or at least a portion of excess heat generated in step c) for removing excess moisture from the biomass feedstock.

In some embodiments, the heat energy generated in step c) is in the form of steam, and the process further comprises recycling at least a portion of the steam to an electricity generator to produce electricity to supplement electricity for the co-electrolyzer.

In some embodiments, the heat energy generated in step e) is in the form of steam, and the process further comprises recycling at least a portion of the steam to an electricity generator to produce electricity to supplement electricity for the co-electrolyzer, and/or to remove excess moisture from the biomass.

In some embodiments, the process further including subjecting the synthesized hydrocarbons to one or more upgrading operations. In some embodiments, a portion of the enhanced hydrogen rich syngas to generate a high purity hydrogen stream. In some embodiments, excess water removed from the biomass is recovered and recycled for supplementing water for generating steam for use in electrolysis step.

A suitable biomass feedstock for the process of the present invention includes, but is not limited to, municipal waste, wood waste, forestry waste material, waste water biomass, municipal sludge, biomass crops such as switchgrass, cattails, and short rotation crops, sewage biomass, agricultural waste (crop residues, livestock by-products, etc.), agricultural by-products, industrial fibrous material, harvested fibrous material or any mixture thereof.

The process of the present invention can incorporate any gasifier known in the relevant art, such as disclosed in U.S. Pat. No. 7,776,114. Preferably, the process of the present invention involves use of the gasifier described in Applicant's PCT Publication No. WO 2018/058252, which is incorporated herein in its entirety.

Examples of suitable FT reactors include fixed bed reactors and slurry-bubble reactors, such as tubular reactors, and multiphase reactors with a stationary catalyst phase.

To gain a better understanding of the invention described herein, the following examples are set forth. It will be understood that these examples are intended to describe illustrative embodiments of the invention and are not intended to limit the scope of the invention in any way.

### EXAMPLES

### Example 1

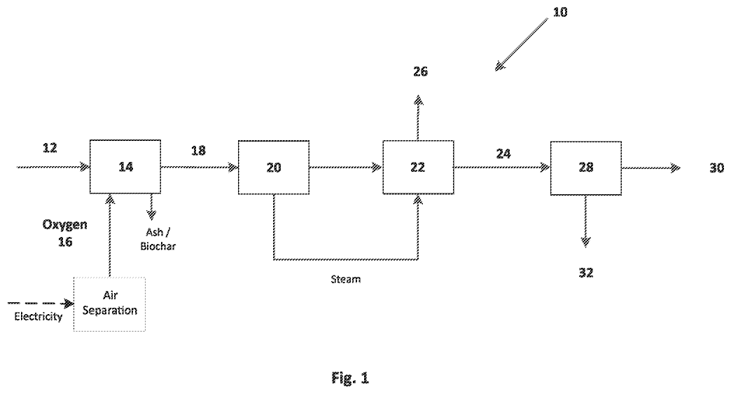

Referring now to FIG. 1, shown is a process flow diagram of a circuit for a prior art process for a gasifying biomass. The process is generally denoted by numeral 10 and begins with a biomass feedstock 12. The biomass is then treated in a gasifier 14 to which oxygen 16 is added as required. As is known, the gasifier generates a hydrogen lean/deficient synthesis gas (syngas) 18 having H₂:CO molar ratio about 1:1, which is optionally subjected to cleaning operations 20 with subsequent water gas shift reaction in unit 22 to form hydrogen rich syngas 24 and carbon dioxide 26, which is rejected to atmosphere or collected.

The hydrogen rich syngas 24 is then transferred to a Fischer-Tropsch reactor 28 to produce the hydrocarbons/FT liquids 30 and process water 32. The resulting hydrocarbons are then passed on to a hydrocarbon cracking stage (not shown) to obtain the desired hydrocarbon products, such as naphtha, diesel etc. The diesel formulated in this process is commonly known as synthetic diesel. In addition, an external source of hydrogen is supplemented to the Fischer-Tropsch unit (not shown) and the hydrocarbon cracking unit.

### Example 2

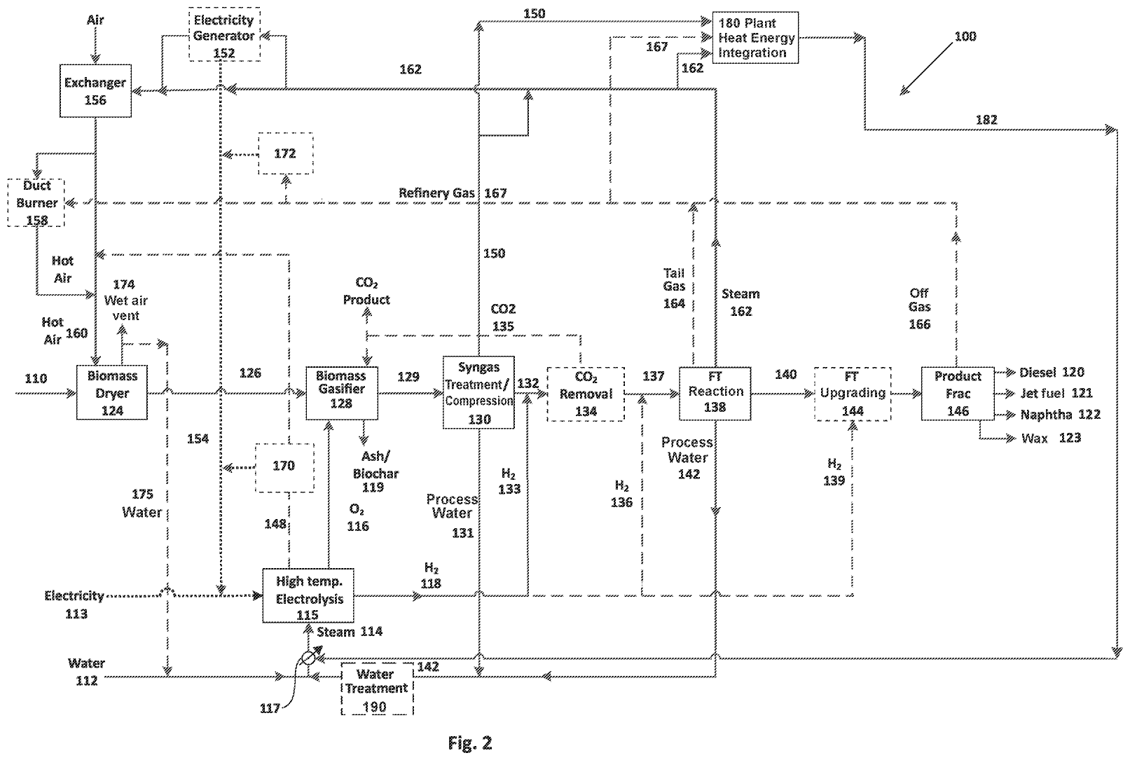

FIG. 2 depicts a flow diagram of an embodiment of the process of the present invention. The process is generally denoted by numeral 100 and begins with electrolysing steam 114 (generated from water 112) with electric power 113, in a high temperature water electrolyzer 115 to generate oxygen 116 and hydrogen 118, and feeding a biomass feedstock 110 to a biomass dryer 124 to remove excess moisture to obtain a dried biomass feedstock 126 having a water content of about 15%. The biomass feedstock 126 and oxygen 116 are then fed to gasifier 128, and the feedstock is gasified under partial oxidation conditions to generate a hydrogen lean syngas 129. The hydrogen lean syngas 129 is subjected to treatment operations 130, such as cooling, condensing, cleaning, compression, etc. to generate process water stream 131 and a cooled raw syngas 132. The cooled raw syngas 132 is optionally subjected to carbon dioxide separation/removal operation 134 to remove CO₂ 135. The CO₂ 135 is optionally fed to the gasifier 128 to be used as blanket/sealing gas.

At least a portion of hydrogen 118 generated via the high temperature electrolysis of steam 114 is added to the hydrogen lean syngas 132 after the syngas treatment operations, via line 133, and/or after carbon dioxide removal operation via line 136, to form hydrogen rich syngas 137. The hydrogen rich syngas 137 is then reacted in a Fischer-Tropsch reactor 138 to produce hydrocarbons 140 and process water stream 142. Hydrocarbons 140 are then subjected to upgrading operation(s) 144, followed by product fractionation 146 to obtain the desired hydrocarbon products, such as diesel 120, jet fuel 121, naphtha 122, wax 123, etc. Optionally a portion of the hydrogen 118 generated vis the electrolysis of steam 114 can be directed via line 139 to the upgrading operations 144. Process water streams 131 and/or 142 are recycled, and used as primary water source for generating steam 114 for the electrolysis step.

Energy/heat from the FT reactor 138 and/or from the biomass gasifier 128 (typically in the form of steam 162 and 150, respectively), and/or refinery gas 167 (such as tail gas from FT reactor, off gas 166 from product fractionation, or both) can be integrated with other plant energy requirements 180, wherein energy/heat 182 from general plant integration 180 can be used to convert water 112 and/or the process water 142 to steam 114 for the electrolysis step.

Alternatively, energy/heat from the FT reactor 138 and/or from the biomass gasifier 128, and/or refinery gas 167 are subjected to heat exchanger 117 to provide heat energy to convert water 112 and/or the process water 142 to steam 114 for the electrolysis step.

Energy/heat from the FT reactor 138 and/or from the biomass gasifier 128 (typically in the form of steam 162 and 150, respectively) can also be used to remove excess moisture from the biomass feedstock, and/or to generate electricity for the electrolysis step.

The steam 162 is passed through heat exchanger 156 to recover heat which is directed as hot air via line 160 to the biomass dryer 124 to supplement the heat used in the excess moisture removal process.

Alternatively, steam from the FT reactor 138 is directed via line 162 to power generator 152 to produce electricity 154 to supplement electricity for the electrolyzer 115, and a portion of the residual steam after power generation is passed through the heat exchanger 156 to recover residual heat which is directed via line 160 to the biomass dryer 124 to supplement the heat used in the excess moisture removal process.

At least a portion of heat generated during the electrolysis process is optionally directed via line 148 to the biomass dryer 124 to supplement the heat used in the excess moisture removal process. In addition, a portion of excess steam generated in the gasifier 128 is optionally directed via line 150 to power generator 152 to produce electricity 154 to supplement electricity for the electrolyzer 115, and a portion of the residual steam after power generation is passed through a heat exchanger 156 to recover heat which is directed via line 160 to the biomass dryer 124 to supplement the heat used in the excess moisture removal process.

At least a portion of water removed from the biomass drier 124 via wet air vent 174 is optionally condensed and recycled as process water via line 175 to generate steam 114 for the steam electrolyzer 115.

In addition, tail gas 164 and/or off gas 166, generated during FT reaction and product fractionation respectively, are used as refinery gas(es) 167 to fire duct burner 158 for biomass dryer 124 to remove excess moisture from the biomass feedstock.

Optionally, a portion of waste heat from the electrolysis step is captured through Organic Rankine Cycle (ORC) and/or Sterling cycle generator 170 to produce electricity 154 to supplement electricity for the electrolyzer 115.

Optionally, the tail gas 164 from the FT reaction and/or the off gas 166 from the fractionation process is used in an internal combustion engine or micro-turbine 172 to generate power for the electrolyzer. The waste heat from the internal combustion engine is captured via ORC technology and/or Sterling cycle generator to produce additional electricity.

### Example 3

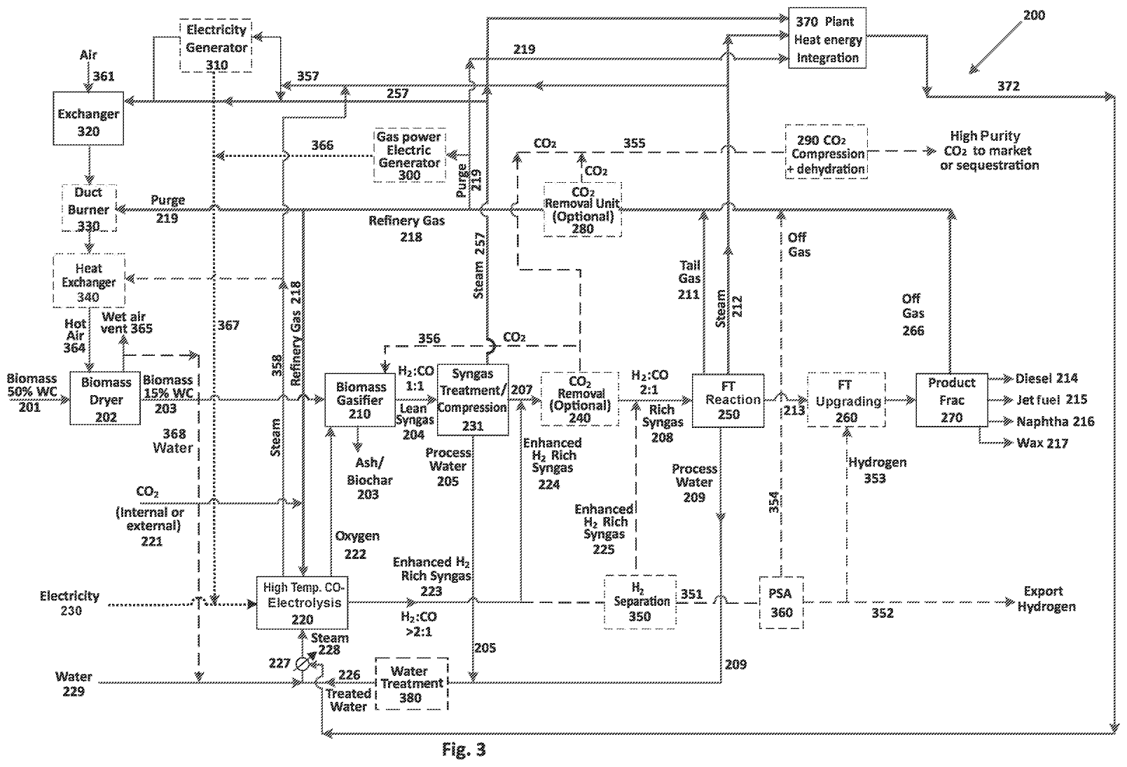

FIG. 3 depicts a flow diagram of another embodiment of the process of the present invention. The process is generally denoted by numeral 200 and begins with electrolysing steam 228 (generated from water 229) and CO₂ 221 with electric power 230, in high temperature co-electrolyzer 220 to generate oxygen 222 and enhanced hydrogen rich syngas 223, and feeding a biomass feedstock 201 to a biomass dryer 202 to remove excess moisture to obtain a drier biomass feedstock 203 having water content about 15%. The dried biomass feedstock 203 and oxygen 222 are then fed to gasifier 210, and the feedstock is gasified under partial oxidation conditions to generate a hydrogen lean syngas 204. The hydrogen lean syngas 204 is subjected to treatment operations 231, such as cooling, condensing, compressing, cleaning, etc. to generate process water stream 205 and a cooled raw syngas 207. The cooled raw hydrogen lean syngas 207 is optionally subjected to carbon dioxide removal operation 240 to remove CO₂ 355. The removed CO₂ 355 is optionally fed to the gasifier 210 to be used as blanket/sealing gas 356.

At least a portion of enhanced hydrogen rich syngas 223 generated via co-electrolysis 220 is added to the hydrogen lean syngas 207 after the cooling and optional cleaning operations via line 224, and/or after carbon dioxide removal operation via line 225, to form hydrogen rich syngas 208. The hydrogen rich syngas 208 is then reacted in a Fischer-Tropsch reactor 250 to produce hydrocarbons 213 and process water stream 209. Hydrocarbons 213 are then subjected to optional upgrading operation(s) in FT upgrader 260, followed by product fractionation 270 to obtain the desired hydrocarbon products, such as diesel 214, jet fuel 215, naphtha 216, wax 217, etc. Process water 209 is optionally subjected to water treatment operation 380, separately or in combination with process water stream 205 to form a treated water stream 226, which may be used as primary water source for generating steam 228 (via heat exchanger 227) for the co-electrolysis step.

Tail gas 211 from the FT reactor 250, off gas from the hydro-processing operation(s) 260, and/or the off gas 266 from the product fractionation 270, are fed as refinery gas stream 218 to the co-electrolyzer 220 with the steam 228 to augment formation of enhanced hydrogen rich syngas. The refinery gas stream 218 is subjected to operations such as compression, heating and/or optional CO₂ removal 280 before being introduced as feed to the co-electrolyzer 220 with the steam 228.

Purge gas streams, such as 219 are removed from the refinery gas stream 218 to reduce/control the concentration of inert compounds/gases in the FT reactor 250. These inert compounds/gases typically include nitrogen and/or argon, which are present in the biomass gasifier oxidant and the biomass. The purge gas 219 may be used as fuel to provide heat energy for general plant use 370, to generate electric power 300 and/or provide heat energy 330 to dry the biomass 201 in the biomass dryer 202. Heat/energy from 372 from general plant integration 370 can be used to convert the water 229 and/or the treated water stream 226 to generate steam 228 for the electrolysis step.

In a further embodiment of the above process the enhanced hydrogen rich syngas 223 has much greater amount of hydrogen than required for optimum hydrogen rich syngas 208, which is achieved by increasing the steam 228 to co-electrolyzer 220 and operating at steam/carbon ratios (S/C) of greater than 2.0 (i.e. 3.0 to 5.0, or 7.0). in such embodiments, the hydrogen separation unit 350, typically consisting of a membrane, PSA unit or absorption unit, is provided to treat the enhanced hydrogen rich stream 223 and produce the optimum enhanced hydrogen rich stream 225 to be combined with the lean hydrogen syngas 207 and a concentrated hydrogen stream 351. The concentrated hydrogen stream 351 can be optionally treated in a PSA unit 360 to produce high purity (>99.9% pure) hydrogen 353 for use in the FT upgrader 260 or marketed as export hydrogen 352. The off gas 354 from the PSA unit 360 is comingled with the refinery gas stream 218 as feed to the co-electrolyzer 220.

High purity bio-CO₂ optionally removed from the CO₂ units 280 and/or 240 is subjected to compression and dehydration 290 for further utilization or sequestration. This bio-CO₂ is recovered from the trees, and therefore this process would directly contribute to the direct reduction of green-house gases (GHG) from the atmosphere.

Energy/heat from the FT reactor 250 and/or from the biomass gasifier 210 (typically in the form of steam 212 and 257, respectively), and/or refinery gas 218 (such as tail gas 211 from FT reactor 250, off gas 266 from product fractionation 270, off gas 354 from hydrogen separation 350 or a combination thereof) can be integrated with other plant energy requirements 370. Heat/energy 372 from general plant integration 370 can be used to convert the water 229 and/or the treated water stream 226 to generate steam 228 for the electrolysis step.

Alternatively, energy/heat from the FT reactor 250 and/or from the biomass gasifier 210, and/or refinery gas 218 are subjected to heat exchanger 227 to provide heat energy to convert water 229 and/or the treated water stream 226 to steam 228 for the electrolysis step.

Energy/heat from the FT reaction 250 and/or from the biomass gasifier 210, (typically in the form of steam 212 and 257, respectively), can also be used to remove excess moisture from the biomass feedstock and/or to generate electricity for the co-electrolyzer 220.

Steam 212 is condensed through heat exchanger 320 to preheat air which is directed via line 364 to the biomass dryer 202 to supplement the heat used to remove the excess biomass moisture.

Alternatively, steam from the FT reactor 212 is directed via line 357 to power generator 310 to produce electricity 367 to supplement electricity 230 for the co-electrolyzer 220, and a portion of the residual steam after power generation is passed through the heat exchanger 320 to recover residual heat which is directed via line 364 to the biomass dryer 202 to supplement the heat used to remove the excess biomass moisture.

At least a portion of heat generated during the co-electrolysis process is optionally directed to the biomass dryer 202 to supplement the heat used to remove the excess biomass moisture, and/or at least a portion of residual steam 358 from the co-electrolysis step directed via line 257 to power generator 310 to produce electricity 367 to supplement electricity for the co-electrolyzer 220, and a portion of the residual steam after power generation is passed through a heat exchanger 320 to recover residual heat, which is optionally directed via line 364 to the biomass dryer 202 to supplement the heat used to remove the excess biomass moisture.

In addition, a portion of excess steam generated in the gasifier 210 is optionally directed via line 357 to power generator 310 to produce electricity 367 to supplement electricity for the co-electrolyzer 220, and a portion of the residual steam after power generation is passed through a heat exchanger 320 to recover residual heat which is directed via line 364 to the biomass dryer 202 to supplement the heat used to remove the excess biomass moisture.

At least a portion of water removed from the biomass drier 202 via wet air vent 365 is optionally condensed and recycled as process water via line 368 to generate steam 228 for the high temperature co-electrolysis 220.

In addition, tail gas 211, generated during FT reaction 250, off gas 266 generated during product fractionation 270 and/or off gas 354 obtained in hydrogen PSA 360 respectively, are used as refinery stream 218 to fire duct burner 330 for biomass dryer 202, thereby using them for removing excess moisture from the biomass feedstock.

Optionally, the tail gas 211, off gas 266 and/or the off gas 354 is used in an internal combustion engine or micro-turbine 300 to generate power for co-electrolyzer 220. The waste heat from the internal combustion engine is captured to produce additional electricity.

### Example 4

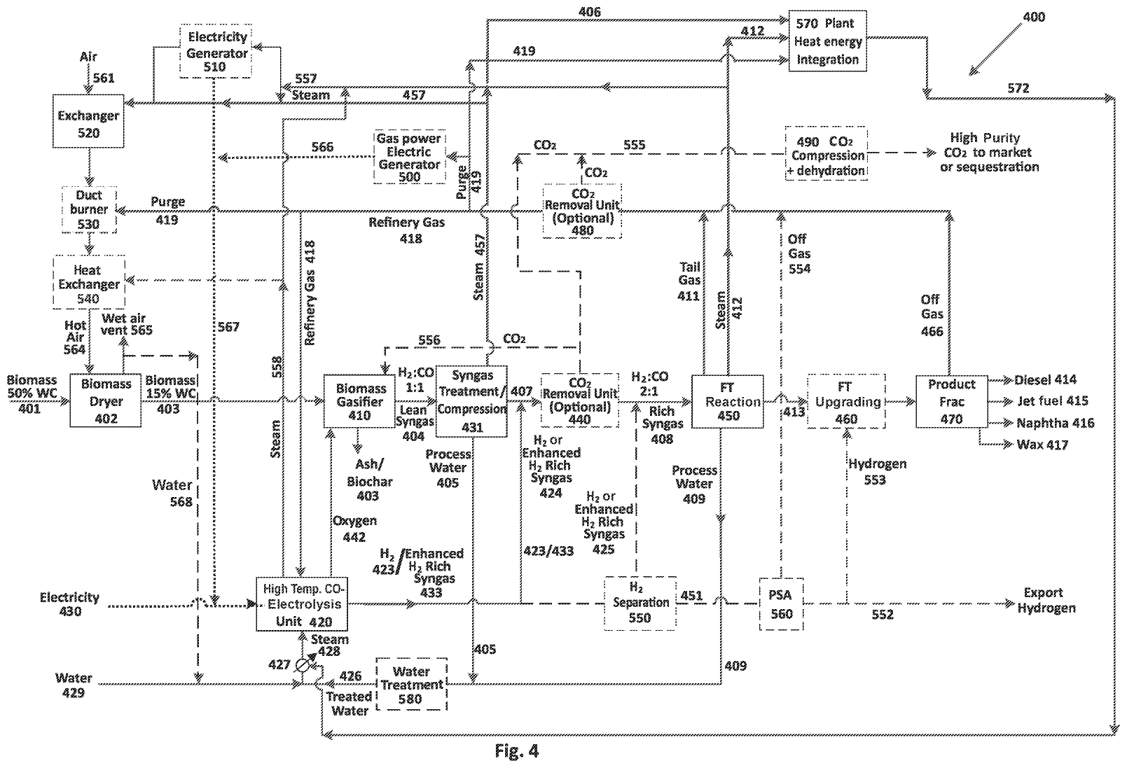

FIG. 4 depicts a flow diagram of another embodiment of the process of the present invention. The process is generally denoted by numeral 400 and begins with electrolysing steam 428 (generated from water 429) with electric power 430, in high temperature electrolyzer 420 to generate oxygen 442 and hydrogen 423, and feeding a biomass feedstock 401 to a biomass dryer 402 to remove excess moisture to obtain a drier biomass feedstock 403 having a water content of about 15%. The dried biomass feedstock 403 and oxygen 422 are then fed to gasifier 410, and the feedstock is gasified under partial oxidation conditions to generate a hydrogen lean syngas 404. The hydrogen lean syngas 404 is subjected to treatment operations 431, such as cooling, condensing, compressing, cleaning, etc. to generate process water stream 405 and a cooled raw syngas 407. The cooled raw syngas 407 is optionally subjected to carbon dioxide removal operation 440 to remove CO₂ 555. The removed CO₂ 555 is optionally fed to the gasifier 410 to be used as blanket/sealing gas 556.

At least a portion of the hydrogen 423 generated via the high temperature electrolysis 420 is added to the hydrogen lean syngas 407 after the treatment operations 431, via line 424, and/or after carbon dioxide removal operation via line 425, to form hydrogen rich syngas 408. The hydrogen rich syngas 408 is then reacted in a Fischer-Tropsch reactor 450 to produce hydrocarbons 413 and process water 409. Hydrocarbons 413 are then subjected to optional upgrading operation(s) in FT upgrader 460, followed by product fractionation 470 to obtain the desired hydrocarbon products, such as diesel 414, jet fuel 415, naphtha 416, wax 417, etc. Process water 409 is optionally subjected to water treatment operation 580, separately or in combination with process water 405 to form a treated water stream 426, which may be used as primary water source for generating steam 428 (via heat exchanger 427) for the electrolysis step.

Optionally, tail gas 411 from the FT reactor 450, off gas from the hydro-processing operation(s) 460 and/or with off gas 466 from the product fractionation 470, are fed as refinery gas stream 418 to the high temperature electrolyzer 420 with the steam 428 to generate enhanced hydrogen rich syngas 433. The refinery gas 418 is subjected to operations such as compression, heating and/or optional CO₂ removal 480 before being introduced as feed to the electrolyzer 420. At least a portion of the enhanced hydrogen rich syngas 433 is then mixed with hydrogen lean syngas 407 to augment formulation of the hydrogen rich syngas 408.

Purge gas streams, such as 419 are removed from the refinery gas stream 418 to reduce/control the concentration of inert compounds/gases in the FT reactor 450. These inert compounds/gases typically include nitrogen and/or argon, which are present in the biomass gasifier oxidant and the biomass. The purge gas 419 may be used as fuel to provide heat energy for general plant use 570, to generate electric power 500 and/or provide heat energy 530 to dry the biomass 401 in the biomass dryer 402.

In a further embodiment of the above process the enhanced hydrogen rich syngas 433 has much greater amount of hydrogen than required for optimum hydrogen rich syngas 408, which is achieved by increasing the steam 428 to electrolyzer 420 and operating at steam/carbon ratios (S/C) of 3.0 to 5.0, or 7.0. in such embodiments, the hydrogen separation unit 550, typically consisting of a membrane, PSA unit or absorption unit, is provided to treat the enhanced hydrogen rich stream 433 and produce the optimum enhanced hydrogen rich stream 425 to be combined with the lean hydrogen syngas 407 and a concentrated hydrogen stream 451. The concentrated hydrogen stream 451 can be optionally treated in a PSA unit 560 to produce high purity (>99.9% pure) hydrogen 553 for use in the FT upgrader 460 or marketed as export hydrogen 552. The off gas 554 from the PSA unit 560 is comingled with the Refinery Gas 418 as feed to the electrolyzer 420.

High purity bio-CO₂ optionally removed from the CO₂ units 480 and/or 440 is treated to compression and dehydration 490 for further utilization or sequestration. This bio-CO₂ is recovered from the trees, and therefore this process would directly contribute to the direct reduction of green-house gases (GHG) from the atmosphere.

Energy/heat from the FT reactor 450 and/or from the biomass gasifier 410 (typically in the form of steam 412 and 457, respectively), and/or refinery gas 418 (such as tail gas 411 from FT reactor 450, off gas 466 from product fractionation 470, off gas 554 from hydrogen separation unit 550, or a combination thereof) can be integrated with other plant energy requirements 570. Heat/energy 572 from general plant integration 570 can be used to convert the water 429 and/or the treated water stream 426 to generate steam 428 for the electrolysis step.

Alternatively, energy/heat from the FT reactor 450 and/or from the biomass gasifier 410, and/or refinery gas 418 are subjected to heat exchanger 427 to provide heat energy to convert the water 429 and/or the treated water stream 426 to generate steam 428 for the electrolysis step.

Energy/heat from the FT reactor 450 and/or from the biomass gasifier 410 (typically in the form of steam 412 and 457, respectively), can also be used to remove excess moisture from the biomass feedstock and/or to generate electricity for the co-electrolyzer 420.

The steam 412 is condensed through heat exchanger 520 to preheat air which is directed via line 564 to the biomass dryer 402 to supplement the heat used to remove the excess biomass moisture.

Alternatively, steam 412 from the FT reactor 450 is directed via line 557 to power generator 510 to produce electricity 567 to supplement electricity 470 for the co-electrolyzer 420, and a portion of the residual steam after power generation is passed through the heat exchanger 520 to recover residual heat which is directed via line 564 to the biomass dryer 402 to supplement the heat used to remove the excess biomass moisture.

In addition, a portion of excess steam generated in the gasifier 410 is optionally directed via line 457 to power generator 510 to produce electricity 567 to supplement electricity for the co-electrolyzer 420, and a portion of the residual steam after power generation is passed through a heat exchanger 520 to recover residual heat which is directed via line 564 to the biomass dryer 402 to supplement the heat used to remove the excess biomass moisture.

At least a portion of heat generated during the electrolysis/co-electrolysis process is optionally directed to the biomass dryer 402 to supplement the heat used to remove the excess biomass moisture, and/or at least a portion of residual steam 558 from the co-electrolysis step is directed via line 557 to power generator 510 to produce electricity 567 to supplement electricity for the co-electrolyzer 420, and a portion of the residual steam after power generation is passed through a heat exchanger 520 to recover residual heat, which is optionally directed via line 564 to the biomass dryer 402 to supplement the heat used to remove the excess biomass moisture.

At least a portion of water removed from the biomass drier 402 via wet air vent 565 is optionally condensed and recycled as process water via line 568 to generate steam for the high temperature electrolyzer 420.

In addition, tail gas 411, generated during FT reaction 450, off gas 466 generated during product fractionation 470 and/or off gas 554 obtained in hydrogen PSA 560, are used to fire duct burner 530 for biomass dryer 402, thereby using them for removing excess moisture from the biomass feedstock.

Optionally, the tail gas 411, the off gas 466 and/or the off gas 554 are used in an internal combustion engine or micro-turbine 500 to generate power for co-electrolyzer 420. The waste heat from the internal combustion engine is captured to produce additional electricity.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1-38. (canceled)

39. A process for preparing synthetic hydrocarbons from a biomass feedstock, comprising:
a) electrolyzing steam and CO2 in a high temperature co-electrolyzer to produce oxygen, enhanced hydrogen rich syngas and heat energy;
b) feeding the oxygen generated in step a), and the biomass feedstock into a gasifier, and gasifying the feedstock under partial oxidation reaction conditions to generate a hydrogen lean syngas, wherein the biomass feedstock optionally undergoes a step of removing excess moisture prior to being fed to the gasifier;
c) cooling the hydrogen lean syngas obtained in step b) to generate process water and heat energy;
d) adding at least a portion of the enhanced hydrogen rich syngas generated in step a) to the hydrogen lean syngas to formulate hydrogen rich syngas; and
e) reacting the hydrogen rich syngas in a Fischer Tropsch (FT) reactor to produce the synthetic hydrocarbons, process water, heat energy and refinery gas.

40. The process of claim 39, further comprising recycling at least a portion of the refinery gas produced in step e) to the co-electrolyzer to augment production of the enhanced hydrogen rich syngas.

41. The process of claim 39, further comprising:
recycling at least a portion of the heat energy produced in step a), produced in step c), produced in step e), or a combination thereof, for generating steam for use in step a);
recycling at least a portion of the process water produced in step c), produced in step e), or both for use in step a); and/or
recycling at least a portion of the heat energy produced in step a), produced in step c), produced in step e), or a combination thereof, and/or for removing excess moisture from the biomass, for generating electric power for use in step a).

42. The process of claim 39, further comprising recycling at least a portion of the refinery gas produced in step e) for removing excess moisture from the biomass, generating electric power for use in step a), or both.

43. The process of claim 39, wherein the hydrogen lean syngas is treated to a carbon dioxide separation operation prior to the reaction in the FT-reactor, and the process further comprises:
i) adding at least a portion of the separated carbon dioxide to the co-electrolyzer, and/or
ii) compressing at least a portion of the separated carbon dioxide to generate high purity carbon dioxide for sequestration or market.

44. The process of claim 39, wherein the refinery gas generated in step e) is treated to a carbon dioxide separation operation, and the process further comprises adding at least a portion of the separated carbon dioxide to the co-electrolyzer, and/or compressing at least a portion of the separated carbon dioxide to generate high purity carbon dioxide for sequestration or market.

45. The process of claim 39, further comprising fractionating the synthesized hydrocarbons, wherein additional refinery gas is generated, and the process further comprises recycling at least a portion of the additional refinery gas:
i) to the co-electrolyzer to augment the production of the enhanced hydrogen rich syngas,
ii) for removing excess moisture from the biomass in step b);
iii) for generating electric power for use in step a); or
iv) a combination thereof.

46. The process of claim 45, wherein the additional refinery gas is treated to a carbon dioxide separation operation, and the process further comprises adding at least a portion of the separated carbon dioxide to the co-electrolyzer, and/or compressing at least a portion of the separated carbon dioxide to generate high purity carbon dioxide for sequestration or market.

47. The process of claim 39, further comprising:
recycling at least a portion of heat energy generated in step a) for removing excess moisture from the biomass feedstock; and/or
recycling at least a portion of excess heat generated in step c) for removing excess moisture from the biomass feedstock.

48. The process of claim 39, wherein the heat energy generated in step c) is in the form of steam, and the process further comprises recycling at least a portion of the steam to an electricity generator to produce electricity to supplement electricity for the co-electrolyzer.

49. The process of claim 39, wherein the heat energy generated in step e) is in the form of steam, and the process further comprises recycling at least a portion of the steam to an electricity generator to produce electricity to supplement electricity for the co-electrolyzer, and/or to remove excess moisture from the biomass.

50. The process of claim 39, further including subjecting the synthesized hydrocarbons to one or more upgrading operations.

51. The process of claim 39, further comprising:
treating a portion of the enhanced hydrogen rich syngas to generate a high purity hydrogen stream; and/or
recovering and recycling excess water removed from the biomass for supplementing water for generating steam for use in step a).

52. A process for preparing synthetic hydrocarbons from a biomass feedstock, comprising:
a) electrolyzing steam in a high temperature co-electrolyzer to produce oxygen, hydrogen, and heat energy;
b) feeding the oxygen generated in step a), and the biomass feedstock into a gasifier, and gasifying the feedstock under partial oxidation reaction conditions to generate a hydrogen lean syngas, wherein the biomass feedstock optionally undergoes a step of removing excess moisture prior to being fed to the gasifier;
c) cooling the hydrogen lean syngas obtained in step b) to generate process water and heat energy;
d) adding at least a portion of the hydrogen generated in step a) to the hydrogen lean syngas to formulate hydrogen rich syngas;
e) reacting the hydrogen rich syngas in a Fischer Tropsch (FT) reactor to produce the biosynthetic hydrocarbons, process water, heat energy and refinery gas; and
f) recycling at least a portion of the refinery gas produced in step e) to the co-electrolyzer to generate enhanced hydrogen rich syngas, and adding a portion of the enhanced hydrogen rich syngas in step d) to augment formulation of the hydrogen rich syngas.

53. The process of claim 52, further comprising adding CO2 to the co-electrolyzer to augment production of the enhanced hydrogen rich syngas.

54. The process of claim 53, wherein the CO2 is from an external source or obtained by treating the hydrogen lean syngas and/or the refinery gas to a carbon dioxide separation operation.

55. The process of claim 54, wherein the process further comprises compressing at least a portion of the separated carbon dioxide to generate high purity carbon dioxide for sequestration or market.

56. The process of claim 52, further comprising recycling at least a portion of the heat energy produced in step a), produced in step c), produced in step e), or a combination thereof, for generating steam for use in step a).

57. The process of claim 52, further comprising:
recycling at least a portion of the process water produced in step c), produced in step e), or both for use in step a);
recycling at least a portion of the process water produced in step c), produced in step e), or both for use in step a); and/or
recycling at least a portion of the heat energy produced in step a), produced in step c), produced in step e), or a combination thereof, and/or for removing excess moisture from the biomass, for generating electric power for use in step a).

58. The process of claim 52, further comprising fractionating the synthesized hydrocarbons, wherein additional refinery gas is generated, and the process further comprises recycling at least a portion of the additional refinery gas:
i) to the co-electrolyzer,
ii) for removing excess moisture from the biomass i;
iii) for generating electric power for use in step a); or
iv) a combination thereof.

59. The process of claim 52, further comprising recycling at least a portion of heat energy generated in step a) for removing excess moisture from the biomass feedstock; and/or
recycling at least a portion of excess heat generated in step c) for removing excess moisture from the biomass feedstock.

60. The process of claim 52, wherein the heat energy generated in step c) is in the form of steam, and the process further comprises recycling at least a portion of steam to an electricity generator to produce electricity to supplement electricity for the co-electrolyzer.

61. The process of claim 52, wherein the heat energy generated in step e) is in the form of steam, and the process further comprises feeding at least a portion of steam to an electricity generator to produce electricity to supplement electricity for the co-electrolyzer and/or to remove excess moisture from the biomass.

62. The process of claim 52, further including subjecting the synthesized hydrocarbons to one or more upgrading operations.

63. The process of claim 52, further comprising:
treating a portion of the enhanced hydrogen rich syngas to generate a high purity hydrogen stream; and/or
recovering and recycling excess water removed from the biomass for supplementing water for generating steam for use in step a).
